(19) United States
(12) Patent Application Publication
(10) Pub. No.: US 2025/0243149 A1
(43) Pub. Date: Jul. 31, 2025

(54) IMPROVED PROCESS FOR RECOVERING N,N-DIMETHYLFORMAMIDE (DMF) FROM AN AQUEOUS PROCESS STREAM

(71) Applicant: BASF AGRO B.V., Arnhem (NL)

(72) Inventors: Chao Wang, Palmyra, MO (US); Sean P. O'Hara, Palmyra, MO (US); Patrick Kelleher, Houston, TX (US); David A. Cortes, Palmyra, MO (US); Erika Maria Scheele, Houston, TX (US); Claudio P. Ribeiro, Houston, TX (US)

(21) Appl. No.: 18/854,589

(22) PCT Filed: Apr. 3, 2023
(86) PCT No.: PCT/EP2023/058602

§ 371 Date: Oct. 7, 2024

Related U.S. Application Data
(60) Provisional application No. 63/330,050, filed on Apr. 12, 2022.

(30) Foreign Application Priority Data
May 24, 2022 (EP) 22175003.7

(51) Int. Cl. C07C 231/24; C07D 239/54

(52) U.S. Cl. CPC C07C 231/24; C07D 239/54

(57) ABSTRACT

The invention relates to an improved process for recovering N,N-Dimethylformamide (DMF) from an aqueous process stream by adjusting the pH, extracting with Chloroform and subsequent distillation/rectification.

The present invention relates to a new and highly efficient process for the production of 3-Phenyluracil intermediates

3-Phenyluracils are important intermediates in the process for the preparation of herbicidally active compounds such as Saflufenacil, an inhibitor of the plant enzyme Protoporphyrinogen Oxidase (PPO).

WO2006/010474 inter alia discloses a process for the preparation of a compound of formula (I), a 3-Phenyluracil, as shown in Scheme 1 below.

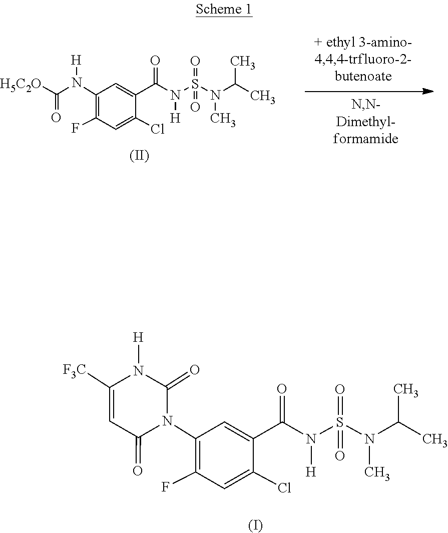

N,N-Dimethylformamide is a suitable solvent in this process. After completion of the reaction, the reaction mixture is added dropwise with cooling to a dilute inorganic acid, such as hydrochloric acid or sulfuric acid. The compound of formula (I) precipitates, is filtered off, washed and dried. The remaining mother liquor (ML) comprises the solvent N,N-Dimethylformamide, water, salts and organic compounds related to the reaction, including byproducts. “Mother liquor” is the term generally used for liquids or solutions remaining after a component has been removed by a process such as crystallization and filtration.

The mother liquor is a waste product, whose adequate disposal is troublesome and costly. For economic and environmental reasons, it is therefore highly desirable to efficiently recover N,N-Dimethylformamide from the mother liquor for reuse in the process according to Scheme 1 or any other chemical process, as appropriate.

N,N-Dimethylformamide (DMF, (CH₃)₂NC(O)H)) is a polar aprotic solvent with a high boiling point (153° C.), a low evaporation rate (low volatility), and high miscibility with water. As a comparably cheap solvent that promotes numerous reactions, DMF has been employed extensively for decades in a variety of chemical processes, ranging from the industrial production of acrylic fibers and plastics to the development and production of biologically active compounds such as pharmaceuticals and pesticides.

In parallel, processes for recovering DMF from process or waste streams, e.g. by extraction and/or distillation/rectification or pervaporation, have been developed to save raw materials, to reduce cost and to minimize environmental impact:

GB1589793 discloses a process for isolating a lower alkylamide such as DMF by extraction from its aqueous solution with an extractant and subsequent distillation of the resulting mixture. According to GB1589793, suitable extractants are substituted phenols of the general formula II

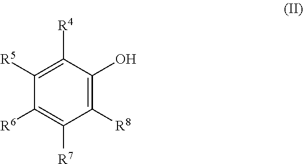

where R⁴ to R⁸ are identical or different and each is hydrogen, alkyl, cyclopentyl or cyclohexyl, with the proviso that the sum of the carbon atoms of R⁴ to R⁵ is from 3 to 9. All phenols according to GB1589793 used as extractant have a boiling point at atmospheric pressure differing from that of DMF by 70° C. to 130° C.

According to Rush, F. et al., methylene chloride is superior to many other solvents examined for isolating lower alkyl amides from aqueous solutions, including carbon tetrachloride, chloroform, trichloroethylene, ethyl acetate, isopropyl ether, butyl acetate, benzene and heptane, Rush, F. E., Olson, J. H., In Smith, B. D., E.I. du Pont de Nemours & Company., & University of Delaware. (1972). DMF recovery and purification: Preliminary design and economic evaluation. Wilmington Del.

CN1317259 discloses a process for recovering dimethylformamide in wastewater by extraction and rectification, using an organic solvent with a boiling point difference to DMF, which is more than or equal to 80° C., e.g. carbon tetrachloride, 1,1 dichloroethane, 1,1,1 trichloroethane, 1,2 dichloroethylene, chloropropane, or trichloropropene.

Chlorinated hydrocarbons like dichloromethane, chloroform, dichloroethane, or carbon tetrachloride were suggested as extractants for DMF from aqueous waste stream in CN101397260, CN106397252, CN108862795, CN108059291, and CN111470997.

In addition to chlorinated hydrocarbons, Dou et al. tested benzene, toluene and long chain alcohols like 1-octanol, 1-nonanol, and 1-decanol as extractants for the removal of DMF from wastewater (Process Safety and Environmental Protection 130 (2019) 317-325).

CN111100028 teaches an extraction-rectification method for DMF using octanoic acid, eugenol, phenol, o-sec-butylphenol or nonylphenol as extractants.

Xiaoyu Wang et al. examined how the presence of salts such as NaCl or Na₂SO₄ affects extraction of DMF with chloroform (J. Chem. Eng. Data 2021, 66, 2233-2243).

Numerous publications teach different solvents to recover DMF from aqueous systems, such as industrial wastewater, however, the results still do not meet today's requirements for economic, safe and practical processes.

Accordingly, it is an object of the present invention to provide a highly efficient process for recovering the solvent N,N-Dimethylformamide in high yield and with high purity from aqueous process or waste streams, which comprise inorganic salts and/or organic compounds. In particular, it is an object of the present invention to provide a highly efficient process for recovering the solvent N,N-Dimethylformamide in high yield and with high purity from aqueous process or waste streams, which comprise inorganic salts and/or high molecular weight organic compounds related to preceding reactions, including byproducts, e.g. the compound of formula (III)

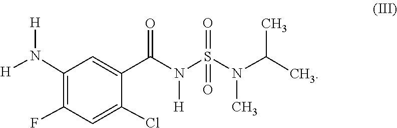 [SMILES: CC(C)N(C)S(=O)(=O)NC(=O)c1cc(N)c(F)cc1Cl]

Furthermore, it is an object of the present invention to provide a highly efficient process for the production of 3-Phenyluracil intermediates, wherein the solvent N,N-Dimethylformamide is recovered in high yield and with high purity from the aqueous mother liquor (ML) after precipitation of the 3-Phenyluracil intermediate. In particular, it is an object of the present invention to provide a highly efficient process for the production of the compound of formula (I)

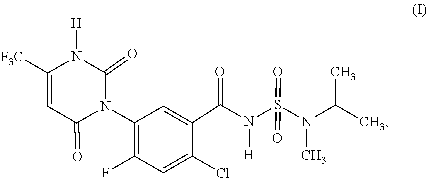 [SMILES: CC(C)N(C)S(=O)(=O)NC(=O)c1cc(-n2c(=O)cc(C(F)(F)F)[nH]c2=O)c(F)cc1Cl]

wherein the solvent N,N-Dimethylformamide is recovered in high yield and with high purity from the aqueous mother liquor (ML) after precipitation of the compound of formula (I). It is a further object of the invention to recover the solvent N,N-Dimethylformamide with such high purity from the aqueous mother liquor, that it can be reused in the process for the production of the compound of formula (I). In particular, it is an object of the invention to recover the solvent N,N-Dimethylformamide from aqueous solutions substantially free of unwanted impurities such as salts and/or high molecular weight organic compounds related to the reaction, including byproducts, e.g. the compound of formula (III)

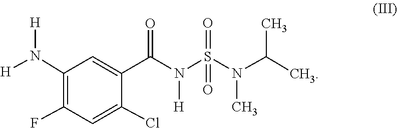 [SMILES: CC(C)N(C)S(=O)(=O)NC(=O)c1cc(N)c(F)cc1Cl]

These and further objectives are achieved by the process described below.

DMF is frequently used as a polar aprotic solvent to promote desired reactions in pesticide manufacturing. Often, the work up of the reaction consists of adding water to precipitate a solid or to extract the product with a water immiscible organic solvent. DMF mostly remains in the aqueous phase and is difficult to recover by distillation since water is lower boiling than DMF and there is usually the presence of a high level of inorganic salts that prevents a DMF recovery of even 50% before salt crystallization occurs. DMF is therefore typically incinerated which contributes to unwanted emissions. Ideally, an organic solvent would extract out all DMF from the aqueous waste stream, leaving the salts and other unwanted impurities such as salts and/or high molecular weight organic compounds related to the reaction, including byproducts, in the aqueous stream with a minimal amount of water in the extracted DMF. The extractant could then be distilled from DMF and recycled, if it has a lower boiling point than DMF, or DMF can be distilled away from a high-boiling solvent. If necessary, DMF could be dried for reuse.

Accordingly, the present invention provides a process for recovering N,N-Dimethylformamide ((CH₃)₂NC(O)H) from aqueous solutions and separating N,N-Dimethylformamide from unwanted impurities in the aqueous solution, comprising the following steps A), B) and C):

### Process Steps A), B) and C):

In a first step A), the pH of the aqueous process stream or the wastewater is adjusted to a basic value. The water content in the aqueous process stream is highly variable, depending on the respective process. Normally, the water content of the aqueous process stream is more than 30% [w/w]. Preferably, the water content of the aqueous process stream is more than 50% [w/w] or 60% [w/w], in particular more than 70% [w/w]. The concentration of DMF in the aqueous process stream is between 1-30% [w/w], preferably between 1-20% [w/w], and most preferably between 5-20% [w/w]. Depending on the respective process, the aqueous process stream may comprise impurities, e.g. inorganic salts or organic compounds related to the reaction. Examples for inorganic salts are alkali metal halides, alkaline earth metal halides, alkali metal sulfates, and alkaline earth metal sulfates, preferably alkali metal halides and alkali metal sulfates such as sodium chloride, potassium sulfate, and sodium sulfate (Na₂SO₄). If present, the concentration of the inorganic salts may be up to 15% [w/w], e.g. between 5 and 15% [w/w] or 7 and 10% [w/w]. The aqueous process stream may comprise organic compounds, e.g. alcohols and other organic solvents, generally in an amount of less than 5% [w/w]. The aqueous process stream may comprise high molecular weight organic compounds related to the reaction, including byproducts. An example for byproducts is the compound of formula (III)

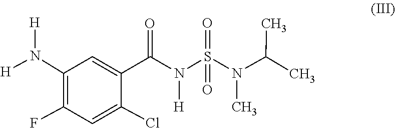 [SMILES: CC(C)N(C)S(=O)(=O)NC(=O)c1cc(N)c(F)cc1Cl]

In the process for the preparation of the compound of formula (I), the compound of formula (III) is the largest higher molecular weight component of the mother liquor after precipitation and filtration of the compound of formula (I). In said process, the mother liquor generally comprises less than 5% [w/w], preferably less than 2% [w/w], more preferably less than 1% [w/w] of the compound of formula (III). In a particular embodiment, the compound of formula (III) is present in the mother liquor in a range between 0 and 0.25% [w/w], more preferably between 0.1 and 0.15% [w/w]. It is important to remove this impurity to minimize accumulation of its level in the process for the preparation of the compound of formula (I) using recycled DMF.

The pH of the aqueous process stream or the wastewater is adjusted to a basic value by adding a suitable inorganic base, e.g. KOH, NaOH, etc., preferably 50% caustic soda. In one embodiment, the pH is adjusted to equal or higher than 8. In another embodiment, the pH is adjusted to equal or higher than 8.5. In another embodiment, the pH is adjusted to equal to or higher than 9. In a preferred another embodiment, the pH is adjusted to equal to or higher than 10.

In a second step B), DMF is extracted from the pH adjusted aqueous process stream with Chloroform (HCCl₃). Under atmospheric pressure, DMF has a boiling point of 153° C., HCCl₃ has a boiling point of 61.2° C. The extraction may be carried out batchwise or continuously, in accordance with conventional techniques and is effected at temperatures from 10 to 60° C., preferably from 20 to 60° C., more preferably from 20 to 30° C. In continuous operation, counter-current extraction is preferred. The weight ratio of the extractant HCCl₃ to the aqueous process stream is from 0.25:1 to 2:1, preferably from 0.5:1 to 2:1, more preferably from 1:1 to 2:1, and in particular 1.5:1. The absolute volumes are variable and depend on the volume of the reaction batch and the equipment. After phase separation, the organic phase is removed. Optionally, the aqueous phase can be extracted with a fresh amount of HCCl₃. The number of washes depends on the weight ratio of extractant and aqueous phase and the desired recovery rate for DMF. Generally, the number of extractions or washes is between 1 and 10, preferably between 2 and 6, more preferably 3 or 4.

If appropriate, the organic phases are combined for subsequent distillation or rectification.

In a third step C), the components of the organic phase from step B) are separated by distillation/rectification in accordance with conventional techniques. Distillation takes place at atmospheric pressure and temperatures between 56-153° C. (top-bottom of tower). Scheme 2 shows a flow diagram of this process.

The organic extract contains almost no salts or water. The chloroform is recovered by distillation for reuse. The chloroform distillation also removes any entrained water and lower boiling components like alcohols such that the recovered DMF can be used directly in a subsequent reaction, e.g. in the process for the preparation of the compound of formula (I).

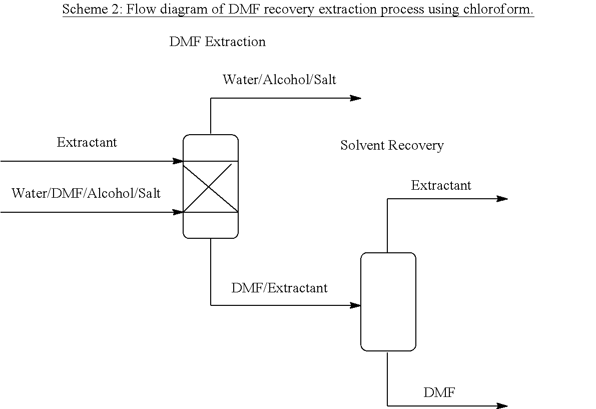

The waste volume is reduced, and the NOX and carbon emissions are greatly reduced when the aqueous phase is incinerated.

### EXAMPLE 1

Chloroform has been identified as a suitable solvent to recover DMF from wastewater streams containing inorganic salts in a liquid-liquid extraction tower. The chloroform to wastewater mass ratio used in the liquid-liquid extraction tower was 1.5:1. Aqueous dimethylformamide (DMF) solution (415 g) containing 10.4% [w/w] of DMF was extracted with 623 g of chloroform in a 1.0 L extraction cell at pH 10 and 40° C. (total volume about 800 ml). The DMF solution to be extracted (ML) originated from a pesticide production waste stream, comprising the compound of formula (III) (cpd. (III)) as high molecular weight impurity. After mixing for 3-5 mins with agitation, the mixture was allowed to settle. Upon phase split, 652 g of organic extract phase containing 4.4% [w/w] of DMF, 0.35% [w/w] of water, 0.38% [w/w] ethanol, and 380 g of raffinate containing 84.4% [w/w] of water, 3.7% [w/w] of DMF, 2.6% [w/w] methanol and 1.2% [w/w] ethanol were obtained. The recovery yield of DMF was 66.8% in a one-stage extraction (see Ex. 1.1 in Table 1 below). These extractions were repeated with fresh chloroform three more times under the same conditions (see Ex. 1.2-1.4 in Table 1 below). The recovery yield of DMF was 98.8% after a four-stage extraction.

TABLE 1

| | # of | HCCl₃:ML | Temp | ML | DMF % | cpd (III)% | H₂O % | |
|---|---|---|---|---|---|---|---|---|
| No. | washes | [w/w] | [° C.] | pH | [w/w] | [w/w]-org | [w/w]-org. | DMF recov. % |
| Ex. 1.1 | 1 | 1.5:1 | 40 | 10 | 66.8 | 0.009 | 0.35 | 66.8% |
| Ex. 1.2 | 2 | 1.5:1 | 40 | 10 | 70.5 | 0.006 | 0.24 | 90.4% ov. 2 stages |
| Ex. 1.3 | 3 | 1.5:1 | 40 | 10 | 67.8 | 0.008 | 0.21 | 96.4% ov. 3 stages |
| Ex. 1.4 | 4 | 1.5:1 | 40 | 10 | 67.9 | 0.004 | 0.21 | 98.9% ov. 4 stages |

A four-stage extraction of the aqueous process stream with a 1.5 to 1 by weight of chloroform removes more than 98% of the DMF from the stream. The raffinate was sent to wastewater and the organic streams were combined and distilled. Distillation took place at atmospheric pressure and temperatures between 56-145° C. The bottoms product was 95.6% pure DMF, containing 3.3% chloroform, which was recycled to the pesticide process without any issue. The distillate was 95% pure chloroform and recycled back to the extraction tower.

### EXAMPLE 2

In analogy to the examples described above, further liquid-liquid extractions with Chloroform were performed. The results are summarized in Tables 2 and 3 below:

TABLE 2

| | | | | | | | | | rel. |
|---|---|---|---|---|---|---|---|---|---|
| | # of | CHCl₃:ML | Temp. | ML | DMF % | cpd (III)% | cpd (III) | cpd (III) | cpd (III) |
| No. | washes | [w/w] | [° C.] | pH | [w/w] | [w/w]-org. | [g]-aqu. | [g]-org. | %-org. |
| Ex. 2.1 | 1 | 2:1 | 40 | 10 | 77.4 | 0.015 | 0.313 | 0.106 | 25.3 |
| Ex. 2.2 | 1 | 1.5:1 | 40 | 10 | 72.2 | 0.009 | 0.380 | 0.057 | 13.0 |
| Ex. 2.3 | 1 | 1:1 | 40 | 10 | 53.4 | 0.011 | 0.461 | 0.056 | 10.8 |
| Ex. 2.4 | 1 | 1.5:1 | 20 | 10 | 68.2 | 0.016 | 0.785 | 0.100 | 11.3 |
| Ex. 2.5 | 1 | 1.5:1 | 50 | 10 | 68.2 | 0.009 | 0.399 | 0.056 | 12.3 |
| Ex. 2.6 | 1 | 1.5:1 | 40 | 10 | 70.4 | 0.01 | 0.378 | 0.063 | 14.3 |

TABLE 3
Comparative Examples at low pH

| | | | | | | | | | rel. |
|---|---|---|---|---|---|---|---|---|---|
| | # of | CHCl₃:ML | Temp. | ML | DMF % | cpd (III)% | cpd (III) | cpd (III) | cpd (III) |
| No. | washes | [w/w] | [° C.] | pH | [w/w] | [w/w]-org. | [g]-aqu. | [g]-org. | %-org. |
| Ex. 2.7 | 1 | 1.5:1 | 40 | 2 | 66.2 | 0.158 | 0.007 | 1.002 | 99.3 |
| Ex. 2.8 | 1 | 1.5:1 | 40 | 7 | 67.4 | 0.041 | 0.115 | 0.260 | 69.3 |
| Ex. 2.9 | 1 | 1.5:1 | 20 | 7 | 74 | 0.033 | 0.134 | 0.209 | 60.9 |

Surprisingly, almost all compound of formula (III) remains in the aqueous layer at high pH. At low pH, it stays with the organic layer. Given that the compound of formula (III) is a weak base, it would be expected to be protonated and more soluble in an acidic aqueous stream.

### EXAMPLE 3

The process according to the present invention was used at pilot scale by continuously extracting DMF from mother liquor (ML) in a Karr extraction column. Chloroform (fresh or recovered by distillation as described above) was used as the extraction solvent. Raffinate from the Karr column was analyzed for remaining DMF, then discarded as waste. The extract from the Karr column was analyzed and separated by distillation. High purity DMF was recovered from the bottoms and chloroform from the overheads as distillate. The DMF and chloroform were recycled a total of five times. For each cycle, 16 L of ML were fed to the extraction column via a 10 L reactor that was heated to 45° C. The Karr extraction column had temperature controlled with jacketed heating or cooling at 45° C. or 20° C. The ML pH was adjusted with 50% caustic soda to the desired value, either pH 8.5 or 10. ML was fed at a rate of 13.7 grams per minute to the bottom of the extraction column and chloroform was fed at a rate of 20.4 grams per minute to the top of the column to achieve counter current flow. The ML contained 14-19% [w/w] DMF and 0.1-0.3% [w/w] compound III, while the chloroform contained 0% [w/w] DMF and 0% [w/w] compound (III). The Karr column had an internal diameter of ⅝ of an inch and a height of 42 inches. The two-phase region of the column was mixed by an internal agitator moving up and down. Aqueous raffinate containing 0.02-0.44% [w/w] DMF eluted at the top of the column and the organic extract containing 8.8-10.4% [w/w] DMF in chloroform eluted at the bottom of the column. After 6 to 9 theoretical stages, DMF recovery was >99%. The results given in Table 4 include temperature, pH, DMF recovery efficiency, mass feed rate of compound (III) in mother liquor, mass flow rate of compound (III) in aqueous raffinate, mass flow rate of compound (III) in organic extract, and percent of compound (III) to be recycled with the extract.

TABLE 4

| | | | ML cpd | Raffinate | Extract | Rel cpd |
|---|---|---|---|---|---|---|
| Extract. | ML | DMF | (III) | cpd (III) | cpd (III) | (III) - |
| Temp ° C. | pH | Rec. % | g/min | g/min | g/min | org % |
| 45 | 10.0 | 99.75% | 3.36 | 2.50 | 0.68 | 20.23% |
| 45 | 10.0 | 99.90% | 3.01 | 2.29 | 0.54 | 17.90% |
| 45 | 8.5 | 99.99% | 3.07 | 1.52 | 1.39 | 45.29% |
| 20 | 10.0 | 99.78% | 3.36 | 2.62 | 0.47 | 14.12% |
| 20 | 8.5 | 99.99% | 3.07 | 1.83 | 1.04 | 33.77% |

### SUMMARY

Surprisingly, the extraction at basic pH values yielded less organic impurity (cpd. (III)) in the organic extract phase in comparison to extraction at neutral or acidic pH values. The percentage of cpd. (III) in the organic extract phase is reduced by 50 to 85% at basic pH values, whereas at pH 2, almost all of cpd. (III) remains in the organic phase. At neutral pH, only 30-40% of cpd (III) are removed. It would have been expected that cpd. (III) as a weak base would be protonated and more soluble in an acidic aqueous stream.

1. A process for recovering N,N-Dimethylformamide (DMF) from an aqueous process stream comprising
A. adjusting a pH of the aqueous process stream to equal or higher than 8;
B. extracting the pH adjusted aqueous process stream with HCCl₃; and
C. recovering the DMF from the organic phase by distillation and/or rectification.

2. The process as claimed in claim 1, wherein the pH of the aqueous process stream is adjusted to equal or higher than 10.

3. The process as claimed in claim 1, wherein a ratio of the HCCl₃ to the aqueous process stream is from 0.25:1 to 2:1.

4. The process as claimed in claim 1, wherein the extraction is carried out at a temperature between 20 to 60° C.

5. The process as claimed in-claim 1, wherein a number of extractions is 3 to 4.

6. The process as claimed in-claim 1, wherein the aqueous process stream comprises a high molecular weight organic impurity.

7. The process as claimed in-claim 1, wherein the aqueous process stream comprises a high molecular weight organic impurity, which is a compound of formula (III)

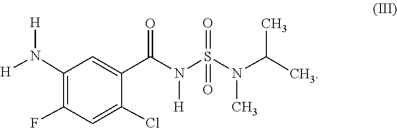 [SMILES: CC(C)N(C)S(=O)(=O)NC(=O)c1cc(N)c(F)cc1Cl]

8. A process for the production of a 3-phenyluracil, wherein a solvent N,N-Dimethylformamide (DMF) is recovered from an aqueous process stream after precipitation and filtration of the 3-phenyluracil comprising the steps of
A. adjusting a pH of the aqueous process stream to equal or higher than 8;
B. extracting the pH adjusted aqueous process stream with HCCl₃; and
C. recovering DMF from the organic phase by distillation and/or rectification.

9. The process as claimed in claim 8, wherein the pH of the aqueous process stream is adjusted to equal or higher than 10.

10. The process as claimed in claim 8, wherein a ratio of HCCl₃ to the aqueous process stream is from 0.25:1 to 2:1.

11. The process as claimed in claim 8, wherein the extraction is carried out at a temperature between 20 to 60° C.

12. The process as claimed in claim 8, wherein a number of extractions is 3 to 4.

13. The process as claimed in claim 8, wherein the aqueous process stream comprises a high molecular weight organic impurity.

14. The process as claimed in claim 8, wherein the aqueous process stream comprises a high molecular weight organic impurity, which is a compound of formula (III)

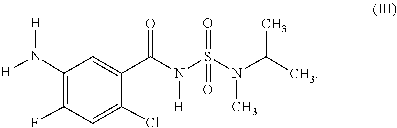 [SMILES: CC(C)N(C)S(=O)(=O)NC(=O)c1cc(N)c(F)cc1Cl]

15. The process as claimed in claim 8, wherein the recovered DMF is reused in the process for the production of the 3-phenyluracil.
